(19) United States
(12) Patent Application Publication
(10) Pub. No.: US 2004/0115227 A1
(43) Pub. Date: Jun. 17, 2004

(54) Thermoformable solid pharmaceutical composition for controlled release of perindopril

(76) Inventors: Patrick Wuthrich, Orleans (FR); Herve Rolland, Olivet (FR); Gilles Briault, Orleans (FR); Gerald Pichon, Orleans (FR); Francois Tharrault, Orleans (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/451,937

(22) PCT Filed: Dec. 21, 2001
(86) PCT No.: PCT/FR01/04133

(30) Foreign Application Priority Data
Dec. 26, 2000 (FR) 00/17013

(51) Int. Cl. A61K 9/00

(52) U.S. Cl. 424/400

(57) ABSTRACT

The present invention relates to a new solid controlled-release pharmaceutical composition obtained by thermoforming, in the hot state, a mixture based on polymers belonging to the polymethacrylate family and perindopril or a pharmaceutically acceptable salt thereof.

The present invention relates to a new solid pharmaceutical composition, for the controlled release of perindopril, obtained by thermoforming, in the hot state, a mixture based on polymer(s) belonging to the polymethacrylate family.

Numerous pharmaceutical compositions intended for the controlled release of pharmaceutical active ingredients have been proposed and produced, for administration by the oral, buccal, sublingual, ocular, rectal, vaginal and/or parenteral routes. The objectives of those new compositions were essentially:

to reduce the frequency of administration of the medicaments,

to obtain relatively constant levels of active ingredient in the target medium or biological site,

to obtain release profiles matching the pharmacological activity of the medicaments.

The principle most commonly employed for controlling release is to incorporate the active ingredient(s), together with excipients, which most often are polymeric in nature, in matrices.

Whatever the matrix compositions envisaged, their production is beset by specific problems of manufacture:

a manufacturing process that is complex and composed of several steps,

stability of the active ingredient during the manufacturing process and in relation to the excipients used,

fine control of the rate of release of the active ingredient(s), which is often variable over time and dependent for example on the particle size of the batches of polymers with the compression processes,

a manufacturing process that allows a pharmaceutical form to be obtained which is essentially suited to just one route of administration,

reproducibility of the batches due to the multiplicity of steps.

Perindopril, the compound of formula:

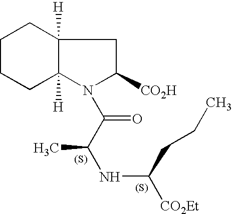

has valuable pharmacological properties. It has, especially, an inhibitory action on certain enzymes, for example carboxypolypeptidases, enkephalinases and kininase II. It inhibits especially the conversion of the decapeptide angiotensin I into the octapeptide angiotensin II—which is, in some cases, responsible for arterial hypertension—by acting on the converting enzyme.

The therapeutic use of perindopril and pharmaceutically acceptable salts thereof makes it possible to reduce, or even suppress, the activity of those enzymes responsible for hypertensive disease and heart failure. The action on kininase II results in an increase in circulating bradykinin and also, by that means, in a reduction in arterial tension.

The tert-butylamine salt of perindopril is currently administered via the oral route in the form of an immediate-release pharmaceutical composition. It has therefore been especially valuable to propose new pharmaceutical compositions that allow the controlled release of that active ingredient.

The object of the present invention is an alternative that, by using techniques of thermoforming, allows the difficulties of a general nature described hereinbefore to be overcome to obtain solid pharmaceutical compositions that allow the controlled release of perindopril and its pharmaceutically acceptable salts. It especially allows the number of steps in the production of final galenical forms to be reduced, thereby limiting the problems of reproducibility and the economic cost, as well as ensuring savings of time and space within a chain of production.

More especially, the invention is directed to a new application of polymethacrylates in the production of the said solid pharmaceutical compositions without addition of plasticiser and without addition of agents that modify the release of the active ingredient(s). The invention, as accomplished by the Applicant, accordingly allows the number of products involved in a galenical formulation to be restricted, thereby limiting the problems of stocking and of supply, as well as the problems associated with management of the environment.

Thermoforming in the hot state relates especially to the techniques of extrusion, co-extrusion, injection and co-injection. These different techniques are well known in the field of plastics and have been widely used in the automobile and packaging sectors.

Because of their characteristics, and the physicochemical properties of the polymers that can be used for thermoforming, the said techniques, and especially simple extrusion, are increasingly being applied in the field of formulating active ingredients.

Various patents accordingly describe controlled-release pharmaceutical compositions which are obtained by extrusion of a mixture comprising at least one active ingredient, one or more extrudable and pharmaceutically acceptable polymers, a plasticiser and/or a retardant, the latter compound allowing the release of the active ingredient to be modified.

In particular, Patent Application WO 96/14058 claims a pharmaceutical composition including especially as therapeutic agent an opioid, which is dispersed in a matrix produced by extrusion. The matrix for extrusion therefore comprises an active ingredient, a hydrophobic material which can be melted, such as an alkylcellulose or an acrylic or methacrylic polymer, and a hydrophobic material, such as a fatty acid or a fatty alcohol. The latter compound serves as retardant and allows the release of the said active ingredient to be slowed down and controlled. A plasticiser is added to the mixture for the purpose of reducing the extrusion temperature.

U.S. Pat. No. 5,102,668 describes a pharmaceutical composition for controlled release which is independent of the pH, the said composition being obtained by wet extrusion of polymers such as polymethacrylates, the said polymers being hydrophilic at low pH and hydrophobic at high pH. The polymethacrylate preferably used is Eudragit® E100. The extrudates thereby obtained must subsequently undergo a spheronisation step and then, advantageously, they are covered with a polymer film composed of Eudragit® NE 30 D. The association between the polymer comprising the extrudate and the polymer comprising the coating film allows the particular technical problem of that invention to be solved, namely control of the release of the active ingredient as a function of the pH of the dissolution medium.

Among the prior art there may also be mentioned the specification DE 41 38 513, which presents a process for the preparation of a controlled-release pharmaceutical composition by continuous extrusion of a mixture comprising at least one active ingredient, a polymethacrylate, and a polymer of N-vinylpyrrolidone and/or of hydroxyalkyl (methyl)cellulose. The latter compounds are used as plasticisers and play a role in regulating the controlled release of the active ingredient.

The article Pharm. Res. 1996, 13 (5), 804-808, also describes the hot extrusion of Eudragit® E100, to which a plasticiser (at least 12% triethyl citrate) has been added, for obtaining films allowing the controlled release of active ingredients.

Similarly, the journals J. Cont. Rel. 1995, 36, 243-250 and Drug Dev. Ind. Pharm. 1994, 20, 1323-1339 report the use of Eudragit® RS PM, to which a plasticiser (triacetin) has been added, for obtaining granules by hot extrusion. The active ingredient release kinetics are rapid and the granules do not release all the active ingredient. The extrusion temperatures are located in the range from 130° C. to 140° C.

Those various documents therefore describe the application of the technique of simple extrusion for obtaining new pharmaceutical compositions. The techniques of injection and co-injection have been much less studied and principally concern solid pharmaceutical compositions wherein the matrix is based on cellulose derivatives, starch or polyethylene glycol.

Finally, with regard to the technique of co-extrusion, Patent Application FR 2 766 088 describes a process for the production of an article from which it is possible to manufacture controlled-release devices, the said process comprising carrying out co-extrusion of polymer and active ingredient, the polymer used being preferably an organosilicate compound capable of cross-linking in the presence or absence of a cross-linking agent.

The present invention allows, in a simple and economical manner, a solid controlled-release pharmaceutical composition to be obtained directly, by simple mixture of perindopril or a pharmaceutically acceptable salt thereof and of polymer(s) that have plastic properties and are pharmaceutically acceptable, without addition of plasticiser or retardant, the said mixture being thermoformed. The continuous control of the release of active ingredient in the said composition is obtained solely by means of judicious selection of the plastic polymer(s) used and of the amount thereof relative to that of the active ingredient. Besides the fact that the pharmaceutical compositions according to the invention are new, they allow galenical forms to be obtained that are easily adaptable to perindopril and pharmaceutically acceptable salts thereof and to their best mode of administration and that ensure controlled and reproducible release thereof.

One of the objects of the invention was to achieve a solid controlled-release pharmaceutical composition comprising a simple mixture of perindopril or a pharmaceutically acceptable salt thereof, and of polymer(s) having plastic properties, the said polymer(s) being composed of the group of the polymethacrylates, without addition of plasticiser and/or retardant, and without use of solvent.

Surprisingly, the solid pharmaceutical compositions of the Applicant can, because of their specific make-up, be subjected to the technique of extrusion, co-extrusion and also of injection or co-injection equally well. Employing the said techniques results in matrices being obtained in forms that have a size and geometry appropriate for various routes of administration, such as, especially, the oral, buccal, sublingual, ocular, vaginal, rectal and parenteral routes. This advantage of the pharmaceutical compositions of the present invention makes it possible to envisage manufacture, starting from the same starting material, of the galenical formulation best suited to the perindopril and pharmaceutically acceptable salt thereof incorporated in the said composition, and to the most appropriate administration route therefor.

Finally, one of the objects of the invention was to obtain a solid pharmaceutical composition wherein it would be possible, by simply adapting the amounts of perindopril and plastic polymers used, to modify the release of perindropril by simple means.

More specifically, the present invention relates to a solid controlled-release pharmaceutical composition, administrable especially by the oral route, comprising a thermoformable mixture of perindopril or a pharmaceutically acceptable salt thereof and one or more polymers selected from the group of the polymethacrylates, the controlled release of the perindopril being ensured solely by the chemical nature, the amount of the polymethacrylate(s) used and the technique employed for manufacture of the said composition.

In the pharmaceutical compositions according to the invention, the perindopril is preferably in the form of the tert-butylamine salt.

A controlled-release pharmaceutical composition is understood to mean release of perindopril over a period of from several minutes (corresponding to immediate release) to a period of more than 20 hours (corresponding to prolonged release), it being possible for the said release to take place in a manner that is delayed in time after administration of the composition. In the case of a delayed-release pharmaceutical composition, the latency time (corresponding to the time between administration of the said composition and release of the active ingredient) can be a period of from 30 minutes to 8 hours, it being possible for the release of the active ingredient thereafter to be immediate release or prolonged release as defined hereinbefore. Within the context of the invention, it is likewise possible for pharmaceutical compositions to be obtained that have a combination of release profiles, for example immediate release of a portion of the active ingredient followed by one or more delayed release(s).

A polymethacrylate is understood to be a copolymer of methacrylic acid corresponding to a fully polymerised copolymer of methacrylic acid and acrylic or methacrylic ester. The said polymethacrylates are commonly referred to by the name Eudragit® and can be presented in the form of a powder or granules.

A thermoformable mixture is understood to be a mixture capable of undergoing transformation under the combined effect of heat and the shearing forces of an endless screw, for example the techniques of extrusion, co-extrusion, injection and co-injection.

Among the various Eudragit® products commercially available, those preferably used within the context of the invention are Eudragit® RL and RS, which refer to copolymers of ammonium methacrylate that consist of fully polymerised copolymers of acrylic acid and methacrylic acid ester having a small amount of quaternary ammonium groups.

The said Eudragit® products correspond to the general formula (I):

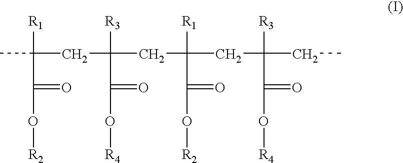

wherein:

R₁ represents a hydrogen atom or a methyl group,

R₂ represents a methyl or ethyl group,

R₃ represents a methyl group,

and R₄ represents a group

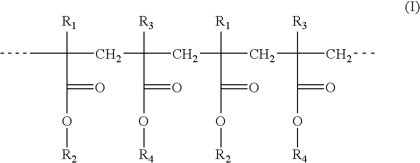

Especially advantageously, the Eudragit® products used in the thermoformable mixture of the invention are Eudragit® RLPO and/or RSPO, which correspond to poly(ethyl acrylate, methyl methacrylate, trimethylaminoethyl methacrylate chloride)'s in the relative proportions of 1:2:0.2 and 1:2:0.1, respectively.

According to another advantageous embodiment of the invention, the thermoformable mixture of the invention can comprise Eudragit® of type E. This polymer corresponds to a poly(butyl methacrylate, (2-dimethylaminoethyl) methacrylate, methyl methacrylate) in the relative proportions of 1:2:1. Eudragit® of type E can be used as the sole polymethacrylate polymer in the thermoformable mixture or can be used in association with Eudragit® RLPO and/or RSPO.

Among the Eudragit® products of type E, special mention may be made of Eudragit® E100, the particular feature of which is that it is soluble at pH's of less than 5, allowing rapid release of the active ingredient in the stomach. As a result of that fact, the use of Eudragit® of type E, and more especially of type E100, is especially well suited to obtaining immediate-release solid pharmaceutical compositions that are administered by the oral route.

According to a third embodiment of the invention, the thermoformable mixture of the invention can comprise Eudragit® of type L100, L100-55 and/or S100. Eudragit® L100 corresponds to a poly(methacrylic acid, methyl methacrylate) in the relative proportions of 1:1. Eudragit® L100-55 corresponds to a poly(methacrylic acid, ethyl acrylate) in the relative proportions of 1:1. Eudragit® S100 corresponds to a poly(methacrylic acid, methyl methacrylate) in the relative proportions of 1:2. These types of Eudragit® can be used as the sole polymethacrylate polymer in the thermoformable mixture or can be used in association with one or more of the other types of Eudragit® mentioned hereinbefore. These polymethacrylates are soluble at pH's of more than 5.5, thereby allowing release of the active ingredient in the intestine and/or colon. Use of the said Eudragit® products is especially valuable in obtaining gastro-resistant solid controlled-release pharmaceutical compositions.

The pharmaceutical compositions thereby obtained within the context of the invention allow, unexpectedly, controlled release of perindopril to be obtained over a period of from several minutes to more than 20 hours, it being possible for that release to be linear, depending on the make-up of the matrix and the technique employed.

The pharmaceutical compositions of the invention are therefore obtained by mixture of perindopril or pharmaceutically acceptable salts thereof and one or more polymethacrylate polymers, lowering of the viscosity of the said mixture under the effect of heat and the shearing forces of an endless screw inside a barrel, and then treatment of the melted mixture by one of the following means:

expulsion from the extruder through a calibrated orifice of variable size and shape, the material obtained being subsequently cut according to the desired final size of the matrix; this constitutes the technique of simple extrusion,

or the first extruder containing the said mixture of reduced viscosity described hereinbefore is associated with a second extruder containing a mixture comprising:

either, solely, one or more polymethacrylate(s) for the control of the release of the active ingredient from the central portion,

or one or more polymethacrylate(s), in admixture with one or more active ingredient(s), which may be the same as or different to that (or those) contained in the central portion,

each extruder operating continuously and feeding the same orifice;

the orifice allows the passage of the mixture coming from the first extruder, ensuring the formation of the inner layer of the final matrix, and also the passage of the mixture coming from the second extruder, ensuring the formation of the outer layer of the final matrix; the extrudate thereby obtained is then cut according to the desired final size and may optionally undergo moulding; the ends of the extrudate may optionally be closed by means of an appropriate technology; this constitutes the technique of co-extrusion;

or injection under pressure, within a press, into moulds having a shape and volume perfectly defined according to the geometric characteristics desired for the matrix; this constitutes the technique of injection;

or the press is equipped with a plurality of injection units allowing injection into one and the same mould, sequentially or simultaneously, of at least two mixtures, which may be the same or different; the first injection unit injects the said mixture, described hereinbefore, which constitutes the central portion, or heart, of the matrix; the second injection unit injects, at the periphery of the central portion, an outer layer of a mixture comprising:

either, solely, one or more polymethacrylate(s) for the control of the release of perindopril or an addition salt thereof,

or one or more polymethacrylate(s), in admixture with perindopril or an addition salt thereof, which may be the same as or different to that (or those) contained in the central portion;

this constitutes the technique of co-injection, which at the same time encompasses the techniques of multi-component injection and of “sandwich” injection.

According to the technique employed, it is therefore possible, within the context of the present invention, to obtain solid controlled-release pharmaceutical compositions that are administrable especially by the oral, buccal, sublingual, ocular, rectal, vaginal or parenteral routes, that are of variable size and geometry, are mono-layered or multi-layered and are best suited to the most appropriate release profiles for perindopril.

The pharmaceutical compositions may be used directly, without another transformation technique being performed apart from packaging. If desired, however, the said pharmaceutical compositions may undergo transformation by grinding or granulation for introduction into a gelatin capsule or for compression or may be subjected to coating.

The pharmaceutical compositions of the invention may optionally also comprise pharmacologically acceptable excipients selected, for example, from the group of anti-oxidants, flavourings, colourings, preservatives, sweeteners and anti-adherents.

The thermoforming temperature is from 60° C. to 150° C. The temperature is preferably from 80° C. to 130° C.

The Examples that follow illustrate the invention but do not limit it in any way.

### EXAMPLE 1

### Extrusion

The compositions of Example 1 are obtained by the technique of extrusion. They all contain 4 mg of perindopril tert-butylamine salt.

The compositions are composed of a mixture comprising 2% perindopril tert-butylamine salt and 98% polymethacrylate.

This Example shows the influence of the nature of the polymethacrylates employed on the in vitro dissolution kinetics of the active ingredient.

The extrusion temperature for the batches comprising Eudragit® RLPO and RSPO is from 105° C. to 110° C. and is 95° C. for the batch comprising Eudragit® E100. Extrusion is carried out using a die 2 mm in diameter, and the speed of the screw is 10 revolutions/min.

TABLE 1
Composition of the test batches

| | Batches | | |
|---|---|---|---|
| Polymethacrylate (98% of the composition) | 1 | 2 | 3 |
| Eudragit ® E100 | 98 | — | — |
| Eudragit ® RLPO | — | 98 | — |
| Eudragit ® RSPO | — | — | 98 |

The in vitro dissolution kinetics were measured by assaying, using high-performance liquid chromatography (HPLC), extrudates containing about 4 mg of perindopril tert-butylamine salt in a buffered dissolution medium of pH 2.

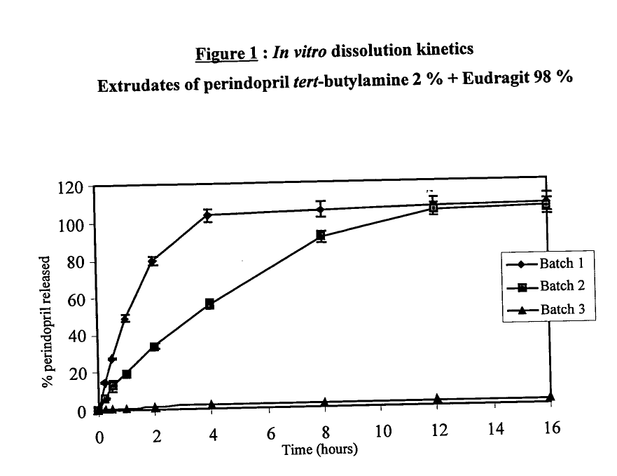

The dissolution kinetics are presented in the annexed FIG. 1.

The results show that, depending upon the type of Eudragit® used, the release kinetics of perindopril tert-butylamine salt can be modified from, for example, 100% of the compound released immediately to 100% of the compound released over 12 hours.

Moreover, and in surprising manner, it has been shown that the perindopril tert-butylamine salt, which is usually unstable to temperature or in association with certain acidic excipients, was not degraded, or only slightly degraded, during extrusion in the hot state carried out at temperatures of from 95° C. to 110° C. (less than 3% degradation). Finally, a stability study over 6 months under variable temperature and relative humidity conditions has demonstrated excellent stability of the perindopril tert-butylamine salt in packaging of the aluminium/PVC blister type.

### EXAMPLE 2

### Co-extrusion

The compositions of this Example were obtained by the technique of co-extrusion. The compositions contain 4 mg of perindopril tert-butylamine salt.

The inner layer is composed of a mixture comprising 4% perindopril tert-butylamine salt and 96% Eudragit® E100. The outer layer is composed of 100% Eudragit® RLPO.

The presence of an outer layer of RLPO polymethacrylate allows the release kinetics of the active ingredient from the inner layer to be slowed down.

The co-extrusion temperatures are 85° C. for the inner layer and from 80 to 105° C. for the outer layer.

The in vitro dissolution kinetics were measured by assaying, using HPLC, extrudates containing 4 mg of perindopril tert-butylamine salt.

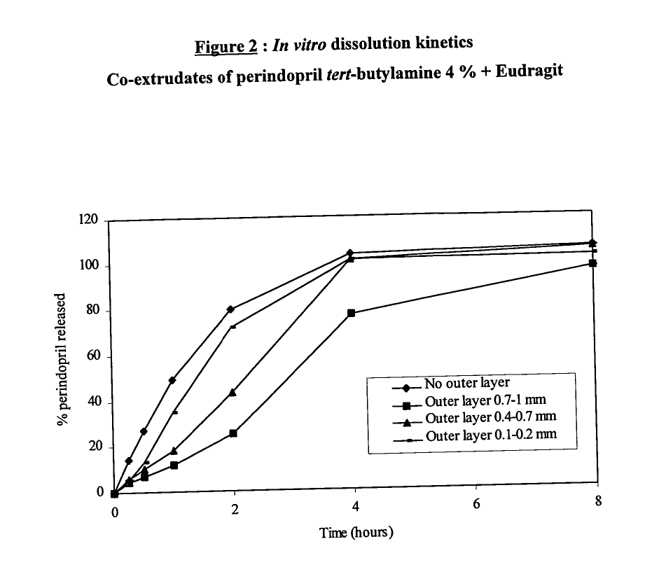

The dissolution kinetics are presented in the annexed FIG. 2.

The results indicate that the addition of an outer layer allows the release kinetics of the perindopril to be made linear and/or to be slowed down: the thicker the outer layer, the more the release kinetics are slowed down.

1- Solid pharmaceutical composition for the controlled release of perindropil or a pharmaceutically acceptable salt thereof, characterised in that it comprises a thermoformable mixture of perindopril or a pharmaceutically acceptable salt thereof and of one or more polymers selected from the group of the polymethacrylates, the release of the perindopril being controlled solely by the nature of the polymethacrylate(s) used, by the amount thereof relative to the perindopril and by the technique employed in the manufacture of the said composition.

2- Solid controlled-release pharmaceutical composition according to claim 1, characterised in that the polymethacrylate(s) used in the thermoformable mixture belong(s) to the family of the Eudragit® products RL and/or RS.

3- Solid controlled-release pharmaceutical composition according to either claim 1 or claim 2, characterised in that the polymethacrylate(s) used in the thermoformable mixture is/are Eudragit® RLPO and/or Eudragit® RSPO.

4- Solid controlled-release pharmaceutical composition according to any one of claims 1 to 3, characterised in that the thermoformable mixture comprises Eudragit® of type E, alone or in association with one or more of the Eudragit® products mentioned hereinbefore.

5- Solid controlled-release pharmaceutical composition according to any one of claims 1 to 4, characterised in that the thermoformable mixture comprises Eudragit® of type L100, L100-55 and/or S100, alone or in association with one or more of the Eudragit® products mentioned hereinbefore.

6- Solid controlled-release pharmaceutical composition according to claim 1, characterised in that the said composition is administrable by one of the routes selected from the oral, buccal, sublingual, ocular, vaginal, rectal and parenteral routes.

7- Solid controlled-release pharmaceutical composition according to any one of claims 1 to 6, characterised in that the said composition is administrable by the oral route.

8- Solid controlled-release pharmaceutical composition according to claim 1, characterised in that the temperature of thermoforming of the mixture is from 60° C. to 150° C.

9- Solid controlled-release pharmaceutical composition according to either claim 1 or claim 8, characterised in that the temperature of thermoforming of the mixture is from 80° C. to 130° C.

10- Solid controlled-release pharmaceutical composition according to claim 1, characterised in that the mixture is thermoformed according to the technique of extrusion.

11- Solid controlled-release pharmaceutical composition according to claim 1, characterised in that the mixture is thermoformed according to the technique of injection.

12- Solid controlled-release pharmaceutical composition according to claim 1, characterised in that the mixture is thermoformed according to the technique of co-extrusion, the inner layer of the said composition in this case being composed of the said mixture and the outer layer of the said composition being composed either of one or more polymethacrylate(s) or of one or more polymethacrylate(s) in admixture with perindopril or a pharmaceutically acceptable salt thereof.

13- Solid controlled-release pharmaceutical composition according to claim 1, characterised in that the mixture is thermoformed according to the technique of co-injection, the central portion of the said composition in this case being composed of the said mixture and the outer layer of the said composition being composed either of one or more polymethacrylate(s) or of one or more polymethacrylate(s) in admixture with perindopril or a pharmaceutically acceptable salt thereof.

14- Solid controlled-release pharmaceutical composition according to claim 1, characterised in that it optionally contains one or more pharmacologically acceptable excipients selected from anti-oxidants, flavourings, colourings, preservatives, sweeteners and anti-adherents.

15- Solid pharmaceutical composition according to claim 1, characterised in that the perindopril is in the form of the tert-butylamine salt.
